(19) United States
(12) Patent Application Publication
(10) Pub. No.: US 2008/0275404 A1
(43) Pub. Date: Nov. 6, 2008

(54) Double Chamber Ampoule

(76) Inventor: Bernd Hansen, Sulzbach-Laufen (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 11/630,987

(22) PCT Filed: Jul. 26, 2005
(86) PCT No.: PCT/EP2005/008093

§ 371 Date: Dec. 28, 2006

(30) Foreign Application Priority Data
Sep. 21, 2004 (DE) 102004046536.3

(51) Int. Cl. A61M 5/31

(52) U.S. Cl. 604/217

(57) ABSTRACT

The invention relates to a receptacle comprised of an elastically flexible plastic material, comprising a dosing chamber (10), inside of which a medium to be discharged is located. One end of the dosing chamber (10) has a discharge opening (18), and the opposite end is connected via a junction point (20) to a compressible receptacle part (22) inside of which an, in particular, gaseous propelling medium is located. When compressing the receptacle part (22), the propelling medium forces, at least in part, the medium to be discharged out of the dosing chamber (10) via the discharge opening (18). An increased functional reliability in every application is ensured by virtue of the fact that the junction point (20) between the dosing chamber (10) and the receptacle part (22) is formed by a bottleneck provided for creating a capillary effect that, independent of the spatial position of the receptacle, prevents a transfer of the medium to be discharged from the dosing chamber (10) into the receptacle part (22) containing the propelling medium.

The invention relates to a receptacle consisting of an elastically flexible plastic material, with a dosing chamber in which a delivery medium is located, one end of the dosing chamber having a discharge opening and the opposite end being connected to a compressible receptacle part via a connecting point, in which there is an especially gaseous propellant medium, when the receptacle part is compressed the propellant medium at least partially displacing the delivery medium out of the dosing chamber via the discharge opening.

DE-PS 32 44 403 discloses a generic receptacle of an elastic plastic material with a tubular dosing chamber in which a material medium is located, and which is made such that the material medium remains in the dosing chamber regardless of the location of the receptacle, and with a compressible receptacle part containing a gaseous medium which has a larger volume than the dosing chamber, and when the receptacle part is compressed the material medium located in the dosing chamber being expellable from this dosing chamber through the discharge opening which can be attached to it.

The known receptacle solution performs the function of delivering a material medium with a consistency that is ointment-like at ambient temperature and that is added exclusively to the dosing chamber when the receptacle is being produced. The consistency of the material medium that is ointment-like at ambient temperature prevents its emergence from the tubular dosing chamber which can also be conical towards the free end of the dosing chamber. In the known receptacle part there is only the gaseous medium as a propellant medium so that when the receptacle is compressed, the material medium which is located in the dosing chamber by itself is removed essentially from this dosing chamber without essentially any residue remaining in the receptacle. The known solution is limited in that for delivery media in the dosing chamber with low viscosity relative to ointment-like consistency the delivery medium from the dosing chamber enters the receptacle part via an enlargement as the connecting point, mixes there with the propellant medium and in this respect renders the receptacle solution unusable.

To counter this disadvantage, according to DE 44 20 594 C2, a plastic receptacle for meterable delivery of flowable substances has been proposed, with a discharge opening provided on one end, and a chamber which is connected to the other open end and which is open only toward the interior of the receptacle, with a volume which can be reduced by manual deformation, and which is made in one piece with the receptacle, the receptacle being made as a cylinder and the interior of the cylinder which holds the flowable substance being separated from the interior of the chamber by a separating piston which can be moved manually lengthwise in the cylinder, in the chamber air being stored which, when displaced by hand, presses the separating piston against the discharge opening of the receptacle. In the known solution the material separation of the delivery medium in the dosing chamber from the propellant medium in the receptacle part is effected by this separating piston, so that mixing does not unintentionally occur; this could make the receptacle solution unusable. With the known receptacle solution it is possible to therefore deliver even a very thin liquid delivery medium, but with the disadvantage that the known solution is expensive to produce due to the separating piston and is axially large, especially in the delivery direction.

On the basis of this prior art, the object of the invention is to devise a receptacle which can be economically produced, which can be made structurally small and which makes it possible to deliver as delivery media those with also a thin liquid consistency and/or in the form of an aerosol without major engineering effort. This object is achieved by a receptacle with the features of claim 1 in its entirety.

In that, as specified in the characterizing part of claim 1, the connecting point between the dosing chamber and the receptacle part is formed from a constriction which is designed such that a capillary action is formed which, independently of the three-dimensional location of the receptacle, prevents overflow of the delivery medium from the dosing chamber into the receptacle part with the propellant medium, it is ensured that even without the separating piston configuration the delivery medium cannot flow unintentionally from the dosing chamber in the direction of the receptacle part with the propellant medium. Regardless of the three-dimensional configuration of the receptacle, operating reliability is thus ensured in each instance and it is therefore surprising for one with average skill in the art in the area of producing these receptacles that he can deliver even sprayable aerosols or the like in this way as the delivery medium. The latter materials are becoming increasingly important in the field of pharmacy since it is possible in this way to bring sprayable active ingredients into contact with the nasal mucosa in order to ensure prompt uptake of the ingredient in this way; this is not ensured in absorption of a different type.

In one preferred embodiment of the receptacle as claimed in the invention, the constriction is formed from a capillary tube. Furthermore provision is preferably made such that the vulnerable constriction is stiffened by a support device. This support device ensures that the constriction cannot be unintentionally damaged or compressed; this could damage its operation.

In one preferred embodiment of the receptacle as claimed in the invention, the support device has at least two crosspiece-like support arms which encompass both at least parts of the receptacle part, the constriction and also the dosing chamber. Preferably the two support arms extend along a separating plane and at least partially overlap it, along which the parts of the receptacle half abut. In addition to the already addressed stiffening of the entire receptacle, this also allows improved sealing of the receptacle contents relative to the exterior so that the sterility of the receptacle interior is also ensured over a longer time.

In another preferred embodiment of the receptacle as claimed in the invention, in an extension of the receptacle part on its side facing away from the dosing chamber the two support arms undergo transition into a tab which is provided with coding. By means of the indicated coding receptacle identification relative to its content can be obtained.

In another, especially preferred embodiment of the receptacle as claimed in the invention, the dosing chamber tapers conically at least in increments toward the discharge opening. Depending on the degree of the selected conical tapering, when the receptacle is compressed an increase in velocity for the stored medium is achieved in this way; this benefits yield. In particular an enhanced jet action can be achieved in delivery.

Other advantageous embodiments of the receptacle as claimed in the invention are the subject matter of the other dependent claims.

The receptacle as claimed in the invention will be detailed below using one embodiment as shown in the drawings. The figures are schematic and not to scale.

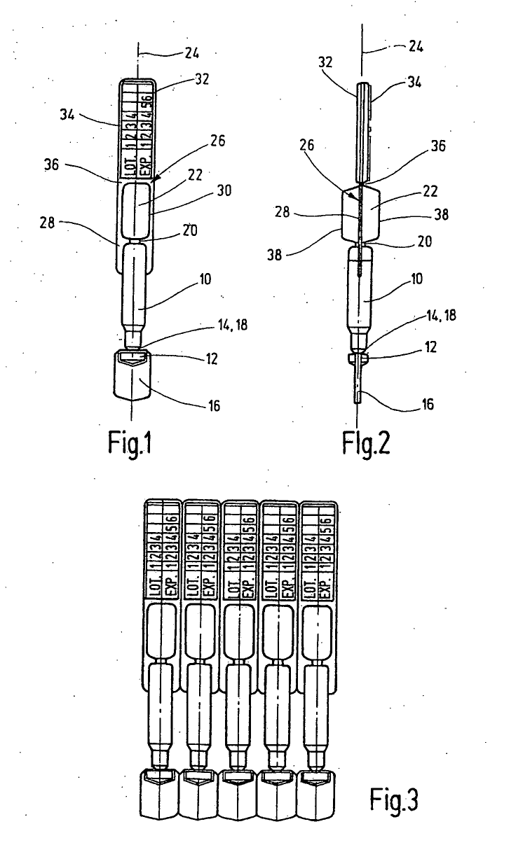

FIG. 1 shows an elevational view of the receptacle as claimed in the invention;

FIG. 2 shows a side view of the receptacle as shown in FIG. 1, and

FIG. 3 shows several separable receptacles which can be combined into a group according to the configuration as shown in FIGS. 1 and 2.

The receptacle shown in the figures consists of a transparent, elastically flexible plastic material which can be produced, filled and sealed sterile, by itself (compare FIGS. 1 and 2) or together with other receptacles (compare FIG. 3) by means of a blow molding machine. The receptacle has a tubular dosing chamber 10 which on its lower end as shown in the drawings is closed by a twist closure 12 which has necked scoring 14 and a handle 16 for screwing off the twist closure 12 in order to thus clear the discharge opening 18 on the lower end of the dosing chamber 10. In the dosing chamber 10 is the delivery medium, for example one with an ointment-like consistency, such as eye ointment; preferably the delivery medium consists however of such a low viscosity, even in the form of an aerosol, that it can be delivered from the dosing chamber 10 via a discharge opening 18 from the receptacle in the form of a spray process. The tubular structure of the dosing chamber 10 would in this way facilitate introduction into body openings, such as the nasal opening or the like.

The opposite end of the dosing chamber 10 is connected via a connecting point 20 to a compressible receptacle part 22 in which an especially gaseous propellant medium, for example in the form of air, is located. When the receptacle part 22 is compressed, for example by hand, the propellant at least partially displaces the delivery medium from the dosing chamber 10 via the discharge opening 18 for an application process outward to the exterior. The connecting point 20 between the dosing chamber 10 and the receptacle part 22 is formed from a constriction as shown in the figures, the constriction being made such that a capillary action is formed which independent of the three-dimension position of the receptacle prevents overflow of the delivery medium from the dosing chamber into the receptacle part 22 with the propellant medium. Likewise the propellant medium cannot unintentionally emerge via the connecting point 20 in the direction of the dosing chamber 10 without compressing the receptacle part 22. In particular the constriction is formed from a capillary tube which is located in the form of a neck in the transition area between the dosing chamber 10 and the receptacle part 22. The axial extension of the capillary tube along the separating plane 24 of the receptacle is in any case smaller than its free passage cross section for the delivery medium.

As is furthermore to be seen in the figures, the vulnerable constriction is stiffened by means of a support device designated as a whole as 26. As shown especially in FIG. 1, the support device 26 has two bridge-like support arms 28, 30 which encompass both the receptacle part 22 and the connecting point 20, as well as at least the lower third of the dosing chamber 10. As is to be seen in FIG. 2 in particular, the two support arms 28, 30 extend along the indicated separating plane 24 and at least partially overlap the plane within the above described framework, so that at the point along which the parts of the receptacle half abut one another in production engineering, additional sealing via the support device 26 is achieved. On its end which is the lower end seen in the direction of looking at FIG. 1, the two support arms 28, 30 undergo transition into one piece into the dosing chamber 10 via a conical admission segment.

In an extension of the container part 22 viewed in the direction of looking at FIGS. 1 and 2 toward the top, the two support arms 28, 30 end in a flat, square tab 32. In addition to improved handling for the receptacle, the tab 32 moreover allows coding 34 to be attached in order in this way to be able to identify the receptacle and its contents. The height of the tab 32 is selected such that as shown in FIG. 2 it projects with an end-side excess end over the separating plane 24 with the support device 26. Optionally the tab 32 could be separated from the receptacle part 22 by way of another scored location 36. In contrast to the dosing chamber 10 which is made tubular, as well as the connecting point 20 in the form of the constriction, the receptacle part 22 is made essentially cuboidal, and, as shown in FIG. 2, on opposing sides has two contact surfaces 38 which enable the receptacle 22 to be is compressed by hand, for example with the thumb and index finger. Furthermore, the contact surfaces 38 in turn extend parallel to the plane of separation 24. In particular, the contact surfaces 38 project distinctly in both directions over the top of the dosing chamber 10. Furthermore, the volumetric ratios of the receptacle part 22 to the dosing chamber 10 are approximately 2:1 in order in this way to be able to ensure complete discharge of the medium via the discharge opening 18.

As shown in FIG. 3, several receptacles on their sides facing one another are connected to one another in succession, the receptacles produced in this way in a series by the blow molding machine being separable from one another via their adjoining faces. In this way, the user is enabled to individually separate and use receptacles combined into groups, preferably a week's supply. This combination permits cutting down on packaging material.

With the receptacle solution as claimed in the invention it can be expected that the possible applications for the described media deliveries will increase for patients.

1. Receptacle consisting of an elastically flexible plastic material, with a dosing chamber (10) in which a delivery medium is located, one end of the dosing chamber (10) having a discharge opening (18) and the opposite end being connected to a compressible receptacle part (22) via a connecting point (20), in which there is an especially gaseous propellant medium, when the receptacle part (22) is compressed the propellant medium at least partially displacing the delivery medium out of the dosing chamber (10) via the discharge opening (18), characterized in that the connecting point (20) between the dosing chamber (10) and the receptacle part (22) is formed from a constriction which is made such that a capillary action is formed which independently of the three-dimensional location of the receptacle prevents overflow of the delivery medium from the dosing chamber (10) into the receptacle part (22) with the propellant medium.

2. The receptacle as claimed in claim 1, wherein the constriction is formed from a capillary tube.

3. The receptacle as claimed in claim 1, wherein the vulnerable constriction is stiffened by means of a support device (26).

4. The receptacle as claimed in claim 3, wherein the support device (26) has at least two bridge-like support arms (28, 30) which encompass both at least parts of the receptacle part (22), the constriction and the dosing chamber (10).

5. The receptacle as claimed in claim 4, wherein the two support arms (28, 30) extend along the separating plane (24), along which parts of the receptacle half abut one another, and at least partially overlap it.

6. The receptacle as claimed in claim 4, wherein in an extension of the receptacle (22) on its side facing away from the dosing chamber (10) the two support arms (28, 30) undergo transition into a tab (32) which is provided with coding (34).

7. The receptacle as claimed in claim 1, wherein the dosing chamber (10) and the constriction are made tubular and the receptacle part (22) is made essentially cuboidal.

8. The receptacle as claimed in claim 7, wherein the cuboidal receptacle part (22) projects at least in one direction over the top of the dosing chamber (10).

9. The receptacle as claimed in claim 1, wherein the ratio of the volumes of the receptacle part (22) to the dosing chamber (10) is at least 2:1.

10. The receptacle as claimed in claim 1, wherein the discharge opening (18) is closed via a twist closure (12) before discharge of the delivery medium.

11. The receptacle as claimed in claim 1, wherein the dosing chamber (10) tapers at least in increments toward the discharge opening (18).
